(19) United States
(12) Patent Application Publication
(10) Pub. No.: US 2017/0147782 A1
(43) Pub. Date: May 25, 2017

(54) METHOD FOR AUTOMATICALLY DETERMINING A CONTRAST AGENT INJECTION PROTOCOL

(71) Applicant: Siemens Healthcare GmbH, Erlangen (DE)

(72) Inventors: Ulrike HABERLAND, Erlangen (DE); Johannes Georg KORPORAAL, Forchheim (DE)

(73) Assignee: Siemens Healthcare GmbH, Erlangen (DE)

(21) Appl. No.: 15/341,319

(22) Filed: Nov. 2, 2016

(30) Foreign Application Priority Data
Nov. 19, 2015 (DE) 102015222853.3

(51) Int. Cl. G06F 19/00; A61M 5/00; A61B 5/055; A61B 6/03; A61B 6/00

(52) U.S. Cl. CPC G06F 19/3443; A61B 6/032; A61B 6/481; A61B 5/055; A61M 5/007; G06F 19/3468; A61M 2205/50

(57) ABSTRACT

A method is disclosed for automatically determining a contrast agent injection protocol for a patient to be examined. An embodiment of the method includes provisioning a plurality of patient reference parameters, a plurality of contrast agent injection protocol parameters and a plurality of assignments of at least one given patient reference parameter with at least one given contrast agent injection protocol parameter; comparing of at least one patient parameter with the plurality of patient reference parameters, the patient parameter being based on patient information; selecting at least one patient reference parameter from the plurality of patient reference parameters based on the comparing; and determining at least one contrast agent injection protocol parameter from the plurality of contrast agent injection protocol parameters.

### PRIORITY STATEMENT

The present application hereby claims priority under 35 U.S.C. §119 to German patent application number DE 102015222853.3 filed Nov. 19, 2015, the entire contents of which are hereby incorporated herein by reference.

### FIELD

At least one embodiment of the invention generally relates to a method for automatically determining a contrast agent injection protocol. Furthermore, at least one embodiment of the invention generally relates to a method for producing a relational database for automatically determining a contrast agent injection protocol. In addition, at least one embodiment of the invention generally relates to a relational database for automatically determining a contrast agent injection protocol. At least one embodiment of the invention further generally relates to a contrast agent injection protocol device for automatically determining a contrast agent injection protocol. In addition, at least one embodiment of the invention generally relates to a medical imaging facility. Finally, at least one embodiment of the invention generally relates to a computer program product with a computer program for automatically determining a contrast agent injection protocol and a computer-readable medium with a computer program for automatically determining a contrast agent injection protocol.

### BACKGROUND

With the aid of modern imaging methods, two or three-dimensional image data which can be used to visualize an imaged object under examination as well as for additional applications is frequently generated.

The imaging methods are frequently based on the recording of X-rays, so-called projection measuring data being generated. For example, projection measuring data can be acquired with the aid of a computed tomography system (CT system). With CT systems, a combination of an X-ray source and an X-ray detector arranged opposite each other on a gantry usually revolves around a measurement space in which the object under examination (which hereinafter is referred to as a patient without loss of generality) is located. The center of rotation (also called the “isocenter”) coincides with a so-called system axis z. In one or more revolutions, the patient is irradiated with X-rays from the X-ray source, projection measuring data and/or X-ray projection measuring data being recorded with the aid of the opposing X-ray detector.

The X-ray detectors used for CT imaging usually have a plurality of detection units which are mostly arranged in the form of a regular pixel array. Each of the detection units generates a detection signal for X-rays striking the detection units which is analyzed at certain times with regard to intensity and spectral distribution of the X-rays in order to draw conclusions about the object under examination and to generate projection measuring data.

Other imaging techniques are based on magnetic resonance tomography. When generating magnetic resonance images, the object under examination is exposed to a relatively high constant magnetic field, for example, of 1.5 Tesla, 3 Tesla, or even 7 Tesla in the case of more recent high magnetic field systems. A high-frequency excitation signal is then emitted using an appropriate antenna device and this results in the nuclear spin of certain resonantly excited atoms in the given magnetic field being tilted by this high-frequency field at a particular flip angle compared to the magnetic field lines of the constant magnetic field. The high-frequency signal emitted during relaxation of the nuclear spin, the so-called magnetic resonance signal, is then picked up using appropriate antenna devices which may also be identical to the transmitting antenna device. The raw data thus acquired is finally used to reconstruct the desired image data. For spatial encoding, respectively defined magnetic field gradients are superimposed on the constant magnetic field during the transmission and readout or receipt of the high-frequency signals.

The aforesaid imaging method can be used to visually reproduce more than just anatomical structures. In addition, work is also increasingly being done on functional imaging by means of the described imaging methods, with which functional and/or dynamic measured variables, such as, for example, measurement of the blood flow rate in blood vessels, can be ascertained.

In the case of the visualization of functional connections as well as body structures of patients, so-called contrast agents are used for medical imaging. However, before contrast-enhanced medical imaging can be started, it must be ensured that after injection of the contrast agent into the body of the patient, the contrast agent is actually present in the area of the patient's body for examination.

This is achieved by coordinated control of the contrast agent injection and image recording. The parameters necessary for this coordinated control can be saved in a contrast agent injection protocol and used for control. A contrast agent injection protocol is a clearly specified period in accordance with which the contrast agent is administered to the patient and comprises, for example, the contrast agent quantity, the starting time, flow rate and end time of administration of the contrast agent. In addition, the contrast agent injection protocol may also comprise the parameters necessary for the control of image recording such as, for example, the time of the image recording, scanning area, scanning duration, and additional parameters of the scan protocol. Contrast agent injection parameters are established on the basis of empirical values and at times adjusted to a specific patient (for example, weight) and according to the kind of examination.

There is the option of making the distribution of the contrast agent visible in the body by performing a so-called bolus tracking scan (BT scan, for short)—this is performed before actual imaging. Such a BT scan may be a time-dependent, low-resolution CT scan with which a time-density curve of a portion of an area for examination is recorded. For a BT scan, such a portion usually comprises a slice which is formed as well as viewed orthogonally to the z-direction, the direction of the system axis of the imaging system. Specifically, in the BT scan attenuation values are recorded as a function of time and space in a portion of the area for examination in which an artery is usually found. If the injected contrast agent now flows through the observed artery, the attenuation values are significantly increased. If a predetermined threshold value of the attenuation values is exceeded, for example, 150 Hounsfield Units (HU), this may be interpreted as evidence that the contrast agent is present in sufficient concentration in the area for examination, and the actual image analysis started. The position and size of the portion examined using the BT scan can usually be changed manually as well.

In the older patent application DE 10 2012 209 410.5, a method for determining an individual, patient-specific contrast agent impulse response function (hereinafter also referred to in short as “impulse response function” or “patient function”) based on test bolus data is described, with which a prediction of the contrast agent course can later be made with a contrast agent-supported imaging measurement. This patient function describes the cardiovascular properties of the patient at the time when the test bolus was measured. In principle, the contrast agent course for any injection protocol could thus be predicted on the assumption that the individual patient function is still valid at the later time.

However, these approaches require the user to have experience or that a test bolus scan is performed and moreover, are not particularly precise. Often, the time at which the image recording is started is scheduled too late, thus extending the overall period for which the contrast agent is in the patient. In principle, however, the aim is to keep the duration of the contrast agent in the body as short as possible because the contrast agent can be detrimental to the human body. If image recording is started too early, this can result in deterioration of the image quality. In the worst-case scenario, image recording and contrast agent administration must even be repeated, constituting an additional strain on the patient.

### SUMMARY

At least one embodiment of the present invention includes developing a more precise and effective method of determining the contrast agent injection protocol in connection with contrast agent imaging.

In general terms, at least one embodiment of the invention relates to a method for automatically determining a contrast agent injection protocol for a patient to be examined which comprises:
a) Provision of a plurality of patient reference parameters, a plurality of contrast agent injection protocol parameters and a plurality of assignments of patient reference parameters to contrast agent injection protocol parameters,
b) Comparison of at least one patient parameter with the plurality of patient reference parameters, the patient parameter being based on patient information,
c) Selection of at least one patient reference parameter from the plurality of patient reference parameters based on step (b),
d) Determination of at least one contrast agent injection protocol parameter from the plurality of contrast agent injection protocol parameters.

At least one embodiment of the invention relates to a method for automatically determining a contrast agent injection protocol for a patient to be examined, comprising:
a) Provision of a relational database having a plurality of patient reference parameters, a plurality of contrast agent injection protocol parameters and a plurality of assignments between at least one given patient reference parameter and at least one given contrast agent injection protocol parameter,
b) Comparison of at least one patient parameter with the plurality of patient reference parameters, the patient parameter being based on patient information,
c) Selection of at least one patient reference parameter from the plurality of patient reference parameters based on step (b),
d) Determination of at least one contrast agent injection protocol parameter from the plurality of contrast agent injection protocol parameters.

Furthermore, at least one embodiment of the invention relates to a method for producing a relational database for automatically determining a contrast agent injection protocol, wherein the database has a plurality of patient reference parameters and a plurality of contrast agent injection protocol parameters, wherein the method comprises:
I. Provision of a plurality of patient reference parameters and a plurality of contrast agent injection protocol parameters,
II. Linking of patient reference parameters and contrast agent injection protocol parameters.

Furthermore, at least one embodiment of the invention relates to a relational database for automatically determining a contrast agent injection protocol, having a plurality of patient reference parameters and a plurality of contrast agent injection protocol parameters, wherein the database has assignments from patient reference parameters and from contrast agent injection protocol parameters.

Furthermore, at least one embodiment of the invention relates to a contrast agent injection protocol device for automatically determining a contrast agent injection protocol for a patient to be examined comprising:
a) a relational database, having a plurality of patient reference parameters, a plurality of contrast agent injection protocol parameters, and a plurality of assignments between at least one given patient reference parameter and at least one given contrast agent injection protocol parameter,
b) an input interface for the entry of a patient parameter based on an item of patient information,
c) a comparison unit for the comparison of the patient parameter with the plurality of patient reference parameters,
d) a selection unit for the selection of a patient reference parameter from the plurality of patient reference parameters based on step (b),
e) a determination unit for the determination of at least one contrast agent injection protocol parameter from the plurality of contrast agent injection protocol parameters.

Furthermore, at least one embodiment of the invention relates to an imaging device, especially a computed tomography system, having a contrast agent injection protocol device according to at least one embodiment of the invention. The imaging device may also have a contrast agent injection device, for example a contrast agent injector. A contrast agent injection device, for example a contrast agent injector, may—in accordance with a contrast agent injection protocol—for example, dispense a defined amount of contrast agent at a defined time, over a defined period, with a defined flow rate and/or a defined flow rate course.

Furthermore, at least one embodiment of the invention relates to a computer program product with a computer program for automatically determining a contrast agent injection protocol which can be loaded directly into a storage device of a control device of an imaging device, preferably of a computed tomography system or a magnetic resonance tomography system, with program sections for all steps of at least one embodiment of the method according to at least one embodiment of the invention when the computer program is executed in the control device of the computed tomography system.

Furthermore, at least one embodiment of the invention relates to a machine-readable medium on which program sections which can be imported and executed by a processor unit are stored in order to execute all the steps of at least one embodiment of the method for automatically determining a contrast agent injection protocol when the program sections are executed by the processor unit.

### BRIEF DESCRIPTION OF THE DRAWINGS

Hereinafter, embodiments of the invention is further explained in example fashion with reference to the example and illustrative figures which show:

FIG. 1 a diagrammatic view of the method according to an embodiment of the invention,

FIG. 2 a diagrammatic view of the medical imaging device according to an embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In general terms, at least one embodiment of the invention relates to a method for automatically determining a contrast agent injection protocol for a patient to be examined which comprises:
a) Provision of a plurality of patient reference parameters, a plurality of contrast agent injection protocol parameters and a plurality of assignments of patient reference parameters to contrast agent injection protocol parameters,
b) Comparison of at least one patient parameter with the plurality of patient reference parameters, the patient parameter being based on patient information,
c) Selection of at least one patient reference parameter from the plurality of patient reference parameters based on step (b),
d) Determination of at least one contrast agent injection protocol parameter from the plurality of contrast agent injection protocol parameters.

At least one embodiment of the invention relates to a method for automatically determining a contrast agent injection protocol for a patient to be examined, comprising:
a) Provision of a relational database having a plurality of patient reference parameters, a plurality of contrast agent injection protocol parameters and a plurality of assignments between at least one given patient reference parameter and at least one given contrast agent injection protocol parameter,
b) Comparison of at least one patient parameter with the plurality of patient reference parameters, the patient parameter being based on patient information,
c) Selection of at least one patient reference parameter from the plurality of patient reference parameters based on step (b),
d) Determination of at least one contrast agent injection protocol parameter from the plurality of contrast agent injection protocol parameters.

A patient parameter is a parameter which can be determined for a patient on the basis of patient information.

According to one embodiment of the invention, the patient parameter is based on patient information which is selected from a group of items comprising:
i. clinical information,
ii. image information, especially image quality information,
iii. patient identity information,
iv. examination archive information, and
v. combinations of (i)-(v).

Patient information may comprise clinical information. The term clinical information encompasses all data and information in relation to the health status of the patient, including age, gender, weight, height, menopausal/hormone status, etiopathological data, medical history data, data collected using in-vitro diagnostic methods, for example blood or urine examinations, data from imaging methods, for example X-ray procedures, CT, MR, SPECT, PET, ultrasound procedures, electrophysiological data, genetic data, gene expression analyses, biopsies, all information from clinical findings including intraoperative and/or interventional findings.

In particular, patient clinical information comprises, for example, age, gender, weight, height, body mass index, blood pressure, heart rate, laboratory findings by way of markers of cardiovascular disease, for example, CRP, BNP, proBNP, NTproBNP, etc., a clinical finding, information with regard to medication of the patient, etc.

The patient parameter may comprise image information, or information derived from image information, for example organ size, vessel diameter, vessel course, etc. The image information may, for example, be obtained using an imaging method or stem from a previous imaging examination. The image information may be static or dynamic image information. The image information may be obtained, for example, by means of a CT scan, MR scan, a CT angiography examination or an ultrasound scan. The image information may comprise image quality information.

The patient parameter may comprise patient identity information, for example patient name, date of birth, insurance number, identity code, etc.

The patient parameter may comprise examination archive information, i.e. information stemming from an earlier examination which, in particular, stems from an earlier imaging examination and which, in particular, stems from an earlier imaging examination with the same image modality for which the contrast agent injection protocol is to be determined.

A patient reference parameter is a parameter which, for example, is stored in the relational database and can be assigned to a patient parameter which can be or was determined by a patient. In this respect, a patient reference parameter can relate to all the parameters to which the patient parameter also relates. In addition, the patient reference parameter can relate to a category of patient parameters, for example an age category, a weight category, a disease category and/or a patient findings category, etc.

The determination of at least one contrast agent injection protocol parameter from the plurality of contrast agent injection protocol parameters takes place on the basis of the selected patient reference parameter.

The relational database comprises a plurality of patient reference parameters, a plurality of contrast agent injection protocol parameters and a plurality of assignments of at least one given patient reference parameter to at least one given contrast agent injection protocol parameter. Patient reference parameters and contrast agent injection protocol parameters can be assigned 1:1, but can also be assigned several times and in a complex manner. A contrast agent injection protocol parameter can thus be assigned to several patient reference parameters. Likewise, a patient-reference parameter can be assigned to several contrast agent injection protocol parameters.

The assignments can be weighted relative to each other, a given first assignment thus being able to have a higher weighting relative to a given second assignment. In this case, for example, the given first assignment would be decisive for the determination of the assigned contrast agent injection protocol parameter with respect to the given second assignment.

According to one embodiment of the invention, it is provided that a plurality of patient parameters is determined and on that basis one or more patient reference parameters are determined.

The method according to at least one embodiment of the invention can be performed by means of a contrast agent injection protocol device which, for example, is designed as a control device for controlling an imaging device 11 and/or on a contrast agent injector.

Furthermore, at least one embodiment of the invention relates to a method for producing a relational database for automatically determining a contrast agent injection protocol, wherein the database has a plurality of patient reference parameters and a plurality of contrast agent injection protocol parameters, wherein the method comprises:
I. Provision of a plurality of patient reference parameters and a plurality of contrast agent injection protocol parameters,
II. Linking of patient reference parameters and contrast agent injection protocol parameters.

According to one embodiment of the invention, it is provided that the step (II) takes place using a machine learning algorithm. To this end, the entries of the plurality of patient reference parameters and a plurality of contrast agent injection protocol parameters can be analyzed using corresponding machine learning algorithms, for example deep learning algorithms. This can take place using a corresponding computer program. This enables the automated generation of new assignments in the database with each data entry.

Furthermore, at least one embodiment of the invention relates to a relational database for automatically determining a contrast agent injection protocol, having a plurality of patient reference parameters and a plurality of contrast agent injection protocol parameters, wherein the database has assignments from patient reference parameters and from contrast agent injection protocol parameters.

Furthermore, at least one embodiment of the invention relates to a contrast agent injection protocol device for automatically determining a contrast agent injection protocol for a patient to be examined comprising:
a) a relational database, having a plurality of patient reference parameters, a plurality of contrast agent injection protocol parameters, and a plurality of assignments between at least one given patient reference parameter and at least one given contrast agent injection protocol parameter,
b) an input interface for the entry of a patient parameter based on an item of patient information,
c) a comparison unit for the comparison of the patient parameter with the plurality of patient reference parameters,
d) a selection unit for the selection of a patient reference parameter from the plurality of patient reference parameters based on step (b),
e) a determination unit for the determination of at least one contrast agent injection protocol parameter from the plurality of contrast agent injection protocol parameters.

Optionally, the contrast agent injection protocol device also has an output interface to output the determined contrast agent injection protocol parameter.

Furthermore, at least one embodiment of the invention relates to an imaging device, especially a computed tomography system, having a contrast agent injection protocol device according to at least one embodiment of the invention. The imaging device may also have a contrast agent injection device, for example a contrast agent injector. A contrast agent injection device, for example a contrast agent injector, may—in accordance with a contrast agent injection protocol—for example, dispense a defined amount of contrast agent at a defined time, over a defined period, with a defined flow rate and/or a defined flow rate course.

Furthermore, at least one embodiment of the invention relates to a computer program product with a computer program for automatically determining a contrast agent injection protocol which can be loaded directly into a storage device of a control device of an imaging device, preferably of a computed tomography system or a magnetic resonance tomography system, with program sections for all steps of at least one embodiment of the method according to at least one embodiment of the invention when the computer program is executed in the control device of the computed tomography system.

Furthermore, at least one embodiment of the invention relates to a machine-readable medium on which program sections which can be imported and executed by a processor unit are stored in order to execute all the steps of at least one embodiment of the method for automatically determining a contrast agent injection protocol when the program sections are executed by the processor unit.

An impending examination can be optimized by the invention using existing data. If the image quality of an earlier examination, for example a CT scan, possibly enhanced with contrast agent, was particularly good, the contrast agent injection protocol parameters can be adjusted to those of the earlier examination to enable particularly good images to be calculated again. The reason for drawing on earlier parameters might be the comparability of the images, for example, in order to assess lesion size under the same injection conditions.

This can be patient-based if the same patient is examined, or also based on population or patient groups if the patient resembles a group of patients for whom data is available. People of a similar weight, with a similar clinical picture, heart rate or a combination of possible groups would be examples.

Relevant information which can be archived from an earlier examination includes, for example, age, habitus, height, weight, scan parameters, all the image data, heart rate, contrast agent protocol, test bolus data and patient response function calculated therefrom (see, for example, DE 10 2012 209 410.5, the entire contents of which are hereby incorporated herein by reference), or bolus tracking data and the image data of the associated CT examination. Derived image information, for example vessel diameters or distances between body regions/organs could also be extracted from existing image information with which the course of contrast agent administration in the body can be predicted/modeled more precisely.

For comparison with existing data or for assignment to a population, all parameters which are available from the contrast agent-enhanced scan, for example information from the RIS (radiology information system), image information from a topogram, age, habitus, height, weight, scan parameters, heart rate, etc. can be used.

The response function of a patient or a group of patients might be used, for example, to calculate a particular enhancement (=enhancement of contrast agents in certain structures) using preset contrast agent injection protocol parameters without a test bolus prior to the scan or to adjust scan and contrast agent injection protocol parameters to achieve the desired enhancement.

Likewise, an existing patient response function might rapidly predict the delay (=temporal interval) until peak enhancement (=maximum enhancement) during a bolus tracking scan. All that would be necessary would be to await the arrival of the contrast agent in the target region, rather than to wait for a certain threshold value to be reached. The invention could also optimize predictions using learning algorithms (machine learning) if the predictions are compared on the basis of the resulting images.

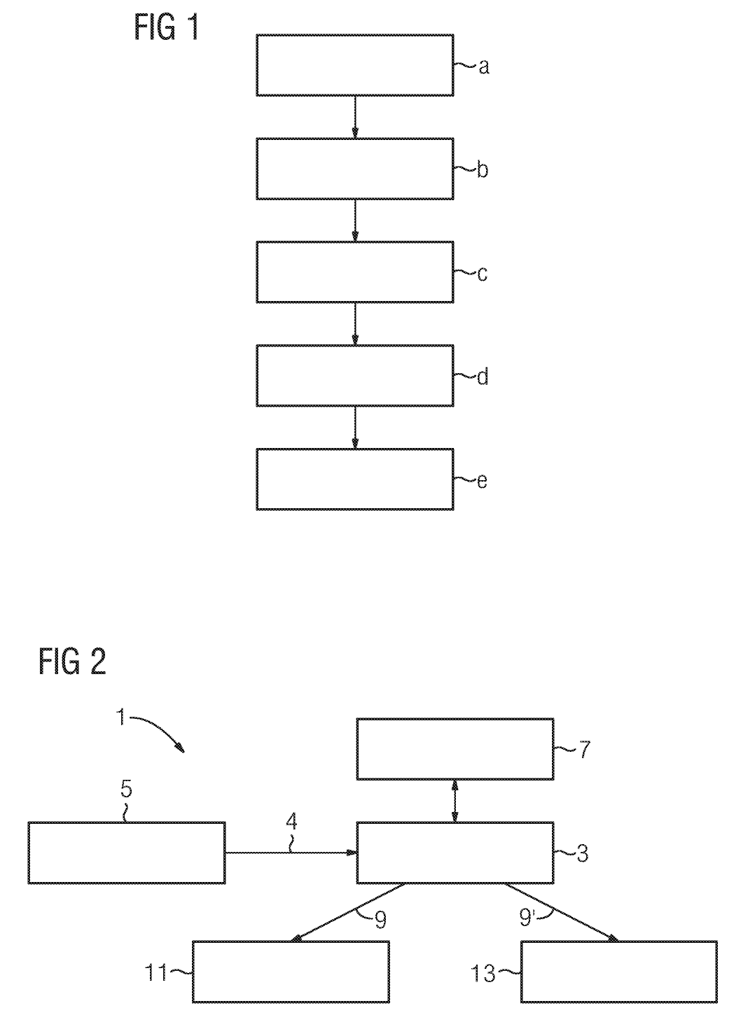

FIG. 1 shows a diagrammatic view of the method according to an embodiment of the invention, wherein a plurality of patient reference parameters, a plurality of contrast agent injection protocol parameters and a plurality of assignments between at least one given patient reference parameter and at least one given contrast agent injection protocol parameter are provided (a). This can, for example, be provided in a corresponding relational database. In accordance with a first example embodiment, a contrast agent injection protocol is to be determined for a 72-year-old patient with a weight of 84 kg and a clinical finding of angina pectoris.

In a further step, the patient parameter is compared with a plurality of patient reference parameters (b). For the automatic determination of a contrast agent injection protocol for a patient to be examined, one or more patient parameters of the patient can be determined on the basis of patient information. To this end, according to the first example embodiment one or more items of clinical information can be determined, for example age, weight, or a clinical finding (i.e. a finding of a particular clinical picture). According to the first example embodiment, this step comprises the classification of the patient to be examined into a patient category from a plurality of patient categories. The patient can, for example, be assigned to a weight category, age category, or a patient finding category (in other words, a category of patient with a finding of a particular clinical picture). Thus, the patient with a weight of 84 kg could be assigned to a weight category of “80 to 90 kg” from the example plurality of weight categories of “<60 kg”, “60-70 kg”, “70-80 kg”, “80-90 kg” or “>90 kg”. In this case, the assigned weight category represents the patient reference parameter. Instead of an individual patient reference parameter, a combination of patient reference parameters can be ascertained, wherein each individual patient reference parameter is selected from a combination of a corresponding plurality of patient reference parameters, for example a weight category of “80 to 90 kg”, an age category of “over 70 years old”, and a patient finding category of “angina pectoris”.

In a further step, selection (c) of the patient reference parameter from the plurality of patient reference parameters takes place on the basis of step (b). According to the first example embodiment, the combination of patient reference parameters is therefore now selected, hence a weight category of “80 to 90 kg”, an age category of “over 70 years old”, and a patient finding category of “angina pectoris”.

In a further step, at least one contrast agent injection protocol parameter is ascertained from the plurality of contrast agent injection protocol parameters (d) based on the selected patient reference parameter. At least one or more contrast agent injection protocol parameters can be ascertained for the selected patient reference parameter or for a plurality or combination of patient reference parameters by way of the given assignments of at least one given patient reference parameter to at least one given contrast agent injection protocol parameter. According to the first example embodiment, in this way the optimum combination of contrast agent injection protocol parameters for a 72-year-old patient with a weight of 84 kg and a clinical finding of angina pectoris can be ascertained. The contrast agent injection protocol comprises the contrast agent injection protocol parameters ascertained in this way.

In a further step (e) the output or forwarding of the one or more ascertained contrast agent injection protocol parameters to an imaging device and/or to a contrast agent injector for the coordinated control of imaging and contrast agent injection then takes place optionally.

According to a further example embodiment, a patient parameter can be determined on the basis of examination archive information, in other words information concerning an earlier examination. The earlier examination may have been carried out on the same patient or on a patient in a corresponding patient category which can also be assigned to the patient to be examined.

According to a further example embodiment, patient identity information, in particular, can be determined as patient information which, for example, enables the identity of the patient to be clearly established. The identity of the patient can be stored as a patient reference parameter, for example in a relational database. Thus, by assigning patient reference parameters to contrast agent injection protocol parameters, it is possible to refer to contrast agent injection protocol parameters from earlier examinations involving the same patient.

According to a further example embodiment, in particular a patient parameter based on image information, in particular image quality information, can be determined. This image information patient parameter can be assigned to a corresponding image information patient reference parameter. Thus, for example, by assigning patient reference parameters to contrast agent injection protocol parameters, it is possible to refer to contrast agent injection protocol parameters from earlier examinations with particularly good image quality.

FIG. 2 shows an imaging device 1. The imaging device 1 may, for example, be a computed tomography device. The imaging device 1 comprises a contrast agent injection protocol device 3, 4, 5, 7, 9, 9′.

The contrast agent injection protocol device comprises a relational database 7 which has a plurality of patient reference parameters, a plurality of contrast agent injection protocol parameters, and a plurality of assignments of patient reference parameters to contrast agent injection protocol parameters.

The contrast agent injection protocol device further comprises an input interface 4, 5 for the input of a patient parameter 5 based on patient information.

The contrast agent injection protocol device comprises a central unit 3 in the example embodiment according to FIG. 2, with a comparison unit for comparing the patient parameter with the plurality of patient reference parameters, a selection unit for the selection of a patient reference parameter from the plurality of patient reference parameters based on step (b) and a determination unit for the determination of at least one contrast agent injection protocol parameter from the plurality of contrast agent injection protocol parameters.

The contrast agent injection protocol device further comprises an output interface 9, 9′ to output the determined contrast agent injection protocol parameter.

Contrast agent injection protocol parameters can, for example, be output via an output interface to an imaging device 11 and/or to a contrast agent injector 13 for the coordinated control of imaging and contrast agent injection. The imaging device may, for example, comprise a computed tomography system and/or device or a magnetic resonance tomography system and/or device.

The method according to an embodiment of the invention can be performed by a contrast agent injection protocol device which, for example, is designed as a control device 3 for the control of an imaging device 11 and/or a contrast agent injector 13.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for automatically determining a contrast agent injection protocol for a patient to be examined, comprising:
provisioning a relational database, including a plurality of patient reference parameters, a plurality of contrast agent injection protocol parameters and a plurality of assignments between at least one given patient reference parameter and at least one given contrast agent injection protocol parameter;
comparing at least one patient parameter with the plurality of patient reference parameters, the patient parameter being based on patient information;
selecting at least one patient reference parameter from the plurality of patient reference parameters based on the comparing; and
determining at least one contrast agent injection protocol parameter from the plurality of contrast agent injection protocol parameters.

2. The method of claim 1, wherein the patient parameter is based on patient information which is selected from a group of items comprising:
i. clinical information,
ii. image information, especially image quality information,
iii. patient identity information,
iv. examination archive information, and
v. combinations of (i)-(v).

3. The method of claim 1, wherein the comparing comprises classifying the patient to be examined into a patient category from a plurality of patients.

4. The method of claim 1, further comprising:
forwarding the at least one contrast agent injection protocol parameter to at least one of an imaging device and to a contrast agent injector.

5. A method for producing a relational database for automatically determining a contrast agent injection protocol, wherein the database includes a plurality of patient reference parameters and a plurality of contrast agent injection protocol parameters, the method comprising:
provisioning a plurality of patient reference parameters and a plurality of contrast agent injection protocol parameters; and
assigning patient R and contrast agent injection protocol parameters.

6. The method of claim 5, wherein the assigning takes place using a machine learning algorithm.

7. A relational database for automatically determining a contrast agent injection protocol, including a plurality of patient reference parameters and a plurality of contrast agent injection protocol parameters, wherein the database includes assignments of patient reference parameters and of contrast agent injection protocol parameters.

8. A contrast agent injection protocol device for automatically determining a contrast agent injection protocol for a patient to be examined, comprising:
a relational database, including a plurality of patient reference parameters, a plurality of contrast agent injection protocol parameters and a plurality of assignments of at least one given patient reference parameter to at least one given contrast agent injection protocol parameter;
an input interface for the input of a patient parameter based on patient information;
a comparison unit for the comparison of the patient parameter with the plurality of patient reference parameters;
a selection unit for the selection of a patient reference parameter from the plurality of patient reference parameters based on the input; and
a determination unit for the determination of at least one contrast agent injection protocol parameter from the plurality of contrast agent injection protocol parameters.

9. The contrast agent injection protocol device of claim 8, further comprising an output interface for output of the determined contrast agent injection protocol parameter.

10. An imaging device, especially a computed tomography system or a magnetic resonance tomography system, comprising a contrast agent injection protocol device as claimed in claim 8.

11. The imaging device of claim 10, further comprising a contrast agent injection device.

12. A non-transitory computer readable medium including a computer program for automatically determining a contrast agent injection protocol, directly loadable into a storage device of a control device of a medical imaging device, to execute the method of claim 1 when the computer program is executed in the control device of the medical imaging device.

13. A non-transitory computer-readable medium including program sections, importable and executable by a processor unit, to execute the method of claim 2 when the program sections are executed by the processor unit.

14. The method of claim 2, wherein the comparing comprises classifying the patient to be examined into a patient category from a plurality of patients.

15. The method of claim 2, further comprising:
forwarding the at least one contrast agent injection protocol parameter to at least one of an imaging device and to a contrast agent injector.

16. The method of claim 3, further comprising:
forwarding the at least one contrast agent injection protocol parameter to at least one of an imaging device and to a contrast agent injector.

17. The method of claim 14, further comprising:
forwarding the at least one contrast agent injection protocol parameter to at least one of an imaging device and to a contrast agent injector.

18. The imaging device of claim 10, wherein the imaging device is a computed tomography system or a magnetic resonance tomography system.

19. The imaging device of claim 11, wherein the imaging device is a computed tomography system or a magnetic resonance tomography system.
